(19) United States
(12) Patent Application Publication
(10) Pub. No.: US 2018/0279886 A1
(43) Pub. Date: Oct. 4, 2018

(54) Method and device for optimizing the measurement accuracy in vivo when measuring invasive blood pressure using a fluid-filled catheter-manometer system

(71) Applicant: INNOVATIVE BUSINESS AND MEDICAL SOLUTIONS, Eke (BE)

(72) Inventor: Erik BILLIET, Eke (BE)

(21) Appl. No.: 15/523,111

(22) PCT Filed: Oct. 27, 2015
(86) PCT No.: PCT/BE2015/000060

§ 371 Date: Apr. 28, 2017

(30) Foreign Application Priority Data
Oct. 28, 2014 (BE) 2014/0751

(51) Int. Cl. A61B 5/0215; A61B 5/00

(52) U.S. Cl. CPC A61B 5/0215; A61B 5/6852; A61B 2562/0247; A61B 5/7271; A61B 2560/0276

(57) ABSTRACT

Method and device for optimizing the measurement accuracy in vivo when measuring invasive blood pressure using a fluid-filled catheter-manometer system including a medical signal processing device; a catheter; a pressure line connected to the catheter; a pressure transducer-flush system unit connected to the pressure line and to a pressurised storage bag, and having an integrated pressure transducer and an integrated flush system, the pressure transducer having a membrane to convert a pressure signal into an electric signal that is transmitted to the medical signal processing device, the flush system ensuring that a continuous flush from the storage bag to the catheter is maintained, the flush system having a manually operable element configured for temporarily, briefly opening and closing the flush system. The following values are calculated: a natural frequency, damping coefficient, a dynamic response diagram using the calculated natural frequency and damping coefficient, and an inverted dynamic response diagram used as a filter to process a signal measured by the pressure transducer; and afterwards, an invasive blood pressure signal and invasive blood pressure values are calculated from the processed signal.

### TECHNICAL FIELD

The invention relates to measuring invasive blood pressure using a fluid-filled catheter-manometer system.

### BACKGROUND ART

A fluid-filled catheter-manometer system comprises: a catheter, filled with a sterile fluid; a pressure line filled with a sterile fluid, having one or more stopcocks and couplings, connected to the outlet of the catheter; a pressure transducer-flush system unit, filled with a sterile fluid, connected to the pressure line and also connected to a pressurised storage bag filled with a sterile fluid; a pressure transducer, integrated into the pressure transducer-flush system unit and provided with a membrane which converts the pressure signal into an electric signal and transmits said electric signal to a medical signal processing device; and a flush system, integrated into the pressure transducer-flush system unit and ensuring that a continuous flushing from the storage bag is maintained, provided with a manually operable element for temporarily briefly opening the flush system and closing it again, or for temporarily opening the flush system for a longer time.

The main field of application is found in departments such as intensive care, operating room, cardiac catheterization and medium care, where for monitoring and therapeutic interventions, multiple hemodynamic parameters are measured continuously. Herein, for measuring invasive blood pressure using a fluid-filled catheter-manometer system, a catheter is inserted in a patient and positioned so that the blood pressure can be measured at the location of interest, commonly the jugular vein, the subclavian vein, the radial artery or the pulmonary artery. The fluid-filled catheter-manometer system is usually connected to a hemodynamic monitor which displays the blood pressure signal, along with its corresponding diastole, systole and mean values, on a screen. An extensive description of the way in which invasive blood pressure is measured—and its medical applications—is found in Manual of Clinical Anesthesiology, Larry F. Chu and Andrea J. Fuller, Wolters Kluwer, Edition 2011, chapters 11-13.

The current state of the art is such that measuring invasive blood pressure is predominantly carried out by means of a fluid-filled catheter-manometer system and not by means of so-called tip transducer systems, due to its cost, its complicated calibration process and its fragile construction. Fluid-filled catheter-manometer systems are therefore widespread, although they do exhibit the property of interfering with the measurement to a certain extent. This interference is mainly due to the fluid-filled part of the catheter-manometer system, as described in Dynamic response of fluid filled catheter systems for measurement of blood pressure: precision of measurements and reliability of the Pressure Recording Analytical Method with different disposable systems, Stefano Romagnoli et al, Journal of Critical Care (2011) 26, 415-422. Its technical feature causes a fluid-filled catheter-manometer system to behave like an underdamped 2nd order measuring system, having as characteristic parameters a natural frequency and a damping coefficient. The physical rules applicable to such a system are described in Dynamic Response of Linear Mechanical Systems—Modeling, Analysis and Simulation, Jorge Angelis, Springer LLC 2011, ISBN 978-1-4419-1026-4. The dynamic response diagram of a fluid-filled catheter-manometer system shows an upswing which is maximalized for the natural frequency of the system. If this upswing is within the bandwidth of the signal to be measured, it leads to an inaccurate measurement. This applies to many catheters and pressure measurement kits currently on the market. This problem is discussed in detail, using as an example arterial blood pressure measurement, in Monitoring Arterial Blood Pressure: What You May Not Know, Beate H. McGhee and Elizabeth J. Bridges, Critical Care Nurse, April 2002 vol. 22 no. 2: 60-79.Also described is how the user should be able to estimate the accuracy of the measurement by interpreting the oscillations following a short pressure pulse applied by means of the flush system. This method is still in use today. Herewith, however, there is no possibility to carry out a correction if the estimate shows that the measurement will not take place with sufficient accuracy. This is a significant drawback, and thus a disadvantage of this method.

On the other hand, a method and device were described in Method and device for removing oscillatory artefacts from invasive blood pressure measurement data, EP 1 769 736 A1, Apr. 4, 2007 Bulletin 2007/14, wherein the natural frequency and damping coefficient are computed from the applied short pressure pulse, after which a recursive algorithm is applied to the distorted blood pressure signal in order to reconstruct the original blood pressure signal. This reconstruction method is very complicated and thus requires an advanced computing unit. Computing times of up to 10 seconds are mentioned. All of this constitutes a major disadvantage of this method.

### DISCLOSURE OF THE INVENTION

It is therefore an aim of the invention to redeem the disadvantages of the above methods and devices so that an optimal accuracy is obtained in vivo when measuring invasive blood pressure using a fluid-filled catheter-manometer system, irregardless of the products that are chosen by the user to build up the fluid-filled catheter manometer system by means of which said measurement is carried out, but also irregardless of any inaccurate filling when installing that system.

To achieve the goal of this invention, a method and device are described wherein a so-called amplifier or also so-called filter is employed which dynamic response diagram is the inverse of the dynamic response diagram of the fluid-filled catheter-manometer system in use. In this way, the upswing typical of the dynamic response diagram of a fluid-filled catheter-manometer system is corrected and a so-called flat dynamic response diagram is obtained, leading to optimal measuring accuracy.

In a preferred embodiment, the method and the device will be implemented in a medical signal processing device serving as a so-called interface between the pressure transducer and a hemodynamic monitor.

In another embodiment, the method and the device will be implemented in the hemodynamic monitor itself.

The invention assumes a fluid-filled catheter-manometer system behaving like an underdamped 2nd order measuring system, wherein the dynamic response diagram can be derived from a step response or from an impulse response.

### BRIEF DESCRIPTION OF THE DRAWINGS

The characteristics and details of the invention will become clear from the following detailed description, referring to the amended drawings, which are an embodiment of the invention provided as a non-limiting example.

FIG. 1 is a general installation scheme according to the invention.

FIG. 2 is an example of a step response in a fluid-filled catheter-manometer system according to FIG. 1.

FIG. 3 is the dynamic response diagram of a fluid-filled catheter-manometer system characterized by a step response according to FIG. 2.

FIG. 4 is the inverted dynamic response diagram of the dynamic response diagram according to FIG. 3.

FIG. 5 is a flat dynamic response diagram.

### MODES FOR CARRYING OUT THE INVENTION

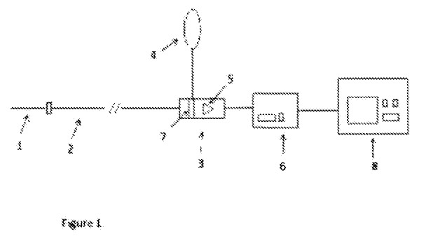

As shown in FIG. 1, the general installation scheme comprises the following: a catheter 1, filled with a sterile fluid, which is positioned inside a patient in such a way that the blood pressure signal to be measured is at the inlet of the catheter 1; a pressure line 2 filled with a sterile fluid, having one or more stopcocks and couplings, connected to the outlet of the catheter 1; a pressure transducer-flush system unit 3, filled with a sterile fluid, connected to the pressure line 2 and also connected to a pressurised storage bag 4 filled with a sterile fluid; a pressure transducer 5, integrated into the pressure transducer-flush system unit 3 and provided with a membrane which converts the pressure signal into an electric signal and transmits said electric signal to a medical signal processing device 6; a flush system 7, integrated into the pressure transducer-flush system unit 3 and ensuring that a continuous flushing from the storage bag 4 to the catheter 1 inlet is maintained, provided with a manually operable element for temporarily briefly opening the flush system and closing it again, or for temporarily opening the flush system for a longer time; and a medical signal processing device 6 serving as an interface between the pressure transducer 5 and a hemodynamic monitor 8.

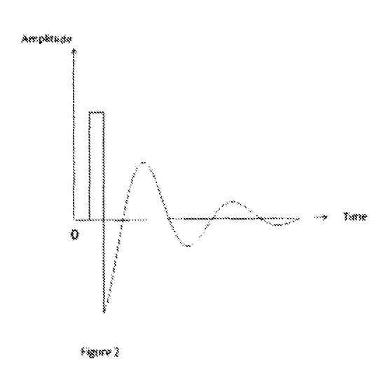

Once the fluid-filled catheter-manometer system is installed on the patient, the user will generate a short pressure pulse in the fluid-filled part of the catheter-manometer system by quickly opening and closing again the flush system 7, after which a damping oscillation will follow, as shown in FIG. 2. By using the applicable physical rules for a step response of an underdamped 2nd order measuring system in the time domain, the medical signal amplifying device 6 calculates the natural frequency and the damping coefficient of the underlying fluid-filled catheter-manometer system.

Using the calculated values of the natural frequency and the damping coefficient, and further using the applicable physical rules for an underdamped 2nd order measuring system in the frequency domain, the medical signal amplifying device 6 then calculates the dynamic response diagram shown, of a system having a response as shown in FIG. 2. The dynamic response diagram of the fluid-filled catheter-manometer system thus presents a typical gain factor in the form of an upswing which indicates certain frequencies being amplified, and therefore incorrectly measured, and wherein the maximum error occurs at the natural frequency of the system.

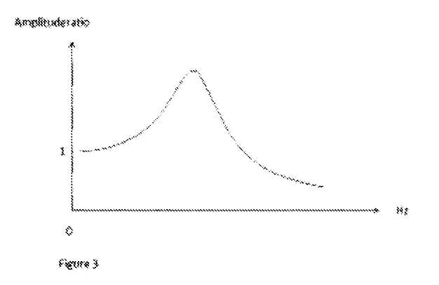

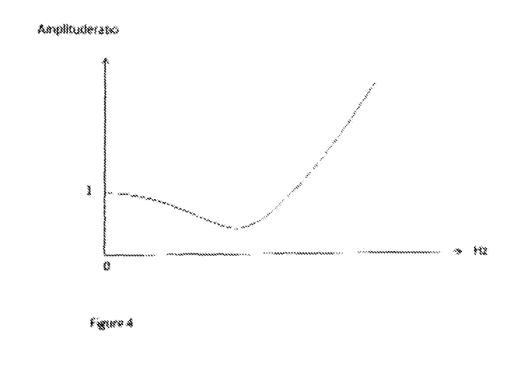

Given the calculated dynamic response diagram, the medical signal amplifying device 6 then calculates the inverted dynamic response diagram by inverting the corresponding gain factor for every frequency. In FIG. 4, the inverted dynamic response diagram of FIG. 3 is shown, implying that it is also the inverted dynamic response diagram of a system having a step response as shown in FIG. 2.

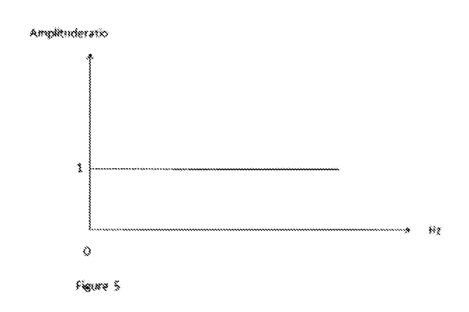

Once this inverted dynamic response diagram is calculated, the medical signal amplifying device 6 will so-called amplify or so-called filter the signal measured by the pressure transducer 5 according to the pattern of the calculated inverted dynamic response diagram. Thus, said signal is processed by the medical signal processing unit 6 and the characteristic upswing in the dynamic response diagram of the fluid-filled catheter-manometer system is fully corrected, leading to a flat dynamic response diagram as shown in FIG. 5. The hemodynamic monitor 8 then further processes said signal for displaying the invasive blood pressure signal, along with its corresponding diastole, systole and mean values and all related calculations. User intervention will thus be limited to applying a short pressure step by means of the flush system 7, wherein estimating the adequacy of the measurement by the user himself will no longer be required, since an optimal measurement accuracy is always achieved by using the method and device of the invention, irregardless of the products used to carry out the invasive blood pressure measurement using a fluid-filled catheter-manometer system, and irregardless of the way said products are installed. This is a significant advantage of the invention in relation to the currently available techniques.

1-4. (canceled)

5. A method for optimizing the measurement accuracy in vivo when measuring invasive blood pressure using a fluid-filled catheter-manometer system comprising:
a medical signal processing device;
a catheter filled with a sterile fluid;
a pressure line filled with a sterile fluid, the pressure line having one or more stopcocks or couplings and being connected to the catheter;
a pressure transducer-flush system unit filled with a sterile fluid, the pressure transducer-flush system unit being connected to the pressure line and to a pressurised storage bag filled with a sterile fluid;
a pressure transducer integrated into the pressure transducer-flush system unit, the pressure transducer being provided with a membrane configured for converting a pressure signal into an electric signal and for transmitting said electric signal to the medical signal processing device; and
a flush system integrated into the pressure transducer-flush system unit, the flush system being configured for ensuring that a continuous flush from the storage bag to the catheter is maintained, the flush system being provided with a manually operable element configured for temporarily, briefly opening the flush system and closing it again, or for temporarily opening the flush system for a longer time,
the method comprising:
calculating a natural frequency and a damping coefficient of the fluid-filled catheter-manometer system;
calculating a dynamic response diagram of the fluid-filled catheter-manometer system by using said natural frequency and said damping coefficient;
calculating an inverted dynamic response diagram of the fluid-filled catheter-manometer system;
using said inverted dynamic response diagram as a filter to process a signal measured by the pressure transducer; and
afterwards, calculating an invasive blood pressure signal and an invasive blood pressure values from the processed signal.

6. The method according to claim 5, wherein the method further comprises hemodynamic monitoring.

7. The method according to claim 5, wherein the dynamic response diagram represents a gain factor for every frequency and in that calculating the inverted dynamic response diagram comprises inverting the gain factor for each frequency.

8. A device for optimizing the measurement accuracy in vivo when measuring invasive blood pressure using a fluid-filled catheter-manometer system, the device comprising:
a medical signal processing device;
a catheter filled with a sterile fluid;
a pressure line filled with a sterile fluid, the pressure line having one or more stopcocks or couplings and being connected to the catheter;
a pressure transducer-flush system unit filled with a sterile fluid, the pressure transducer-flush system unit being connected to the pressure line and to a pressurised storage bag filled with a sterile fluid;
a pressure transducer integrated into the pressure transducer-flush system unit, the pressure transducer being provided with a membrane configured for converting a pressure signal into an electric signal and for transmitting said electric signal to the medical signal processing device; and
a flush system integrated into the pressure transducer-flush system unit, the flush system being configured for ensuring that a continuous flush from the storage bag to the catheter is maintained, the flush system being provided with a manually operable element configured for temporarily, briefly opening the flush system and closing it again, or for temporarily opening the flush system for a longer time,
the medical signal processing device being configured for:
calculating a natural frequency and a damping coefficient of the fluid-filled catheter-manometer system;
calculating a dynamic response diagram of the fluid-filled catheter-manometer system by using said natural frequency and said damping coefficient;
calculating an inverted dynamic response diagram of the fluid-filled catheter-manometer system using said dynamic response diagram;
using said inverted dynamic response diagram as a filter to process a signal measured by the pressure transducer; and
afterwards, calculating an invasive blood pressure signal and an invasive blood pressure values from the processed signal.

9. The device according to claim 8, wherein the device further comprises a hemodynamic monitor.

10. The device according to claim 7, wherein the dynamic response diagram represents a gain factor for every frequency and in that the medical signal processing device is further configured for inverting the gain factor for each frequency to calculate the inverted dynamic response diagram.

11. The device according to claim 8, wherein the catheter has multiple lumina thereby forming multiple fluid-filled catheter-manometer systems.

12. Device according to claim 8, wherein at least one blood collection system is located inside the pressure line.

13. The device according to claim 8, wherein the medical signal processing device is an interface between the pressure transducer and a hemodynamic monitor.

14. The device according to claim 8, wherein the medical signal processing device is integrated in a hemodynamic monitor.
